(19) United States
(12) Patent Application Publication
(10) Pub. No.: US 2024/0420089 A1
(43) Pub. Date: Dec. 19, 2024

(54) BUSINESS-TO-BUSINESS SYSTEM FOR PROMOTING AND OPTIMIZING DIVERSITY IN A CLINICAL OR MEDICAL RESEARCH SETTING

(71) Applicant: Ruqayyah Abdulrahoof, Davie, FL (US)

(72) Inventor: Ruqayyah Abdulrahoof, Davie, FL (US)

(21) Appl. No.: 18/209,028

(22) Filed: Jun. 13, 2023

(51) Int. Cl. G06Q 10/1053; G16H 40/20

(52) U.S. Cl. CPC G06Q 10/1053; G16H 40/20

(57) ABSTRACT

A B2B marketplace software system for increasing diversity among the clinical research and development workforce and trust within diverse communities resulting in diverse clinical trials is disclosed. The B2B marketplace software system is configured to connect biotechnology/biopharmaceutical/medical device/health tech and other relevant companies with a pool of qualified clinical research suppliers; connect a diverse pool of qualified talent with biotechnology/biopharmaceutical/medical device/health tech and other relevant companies; cut talent recruitment and supplier discovery costs for biotechnology/biopharmaceutical/medical device/health tech and other relevant companies; and connect industry suppliers with more opportunities.

### TECHNICAL FIELD

The present disclosure relates, in general, to business-to-business software, and, more particularly, but without limitation, to business-to-business online software utilized to engage in exchanges between professionals and organizations in the healthcare, medical, clinical research or health technology spaces.

### BACKGROUND

Software systems for evaluating corporate responsibility and cultural well-being are known in the art. However, none of the art provides the lack of diversity in clinical research and development and creating trust in communities. There is a need for a business-to-business (“B2B”) method of hiring diverse candidates in a clinical research setting in order to improve trust among diverse populations.

### SUMMARY

This summary is provided to introduce a variety of concepts in a simplified form that is disclosed further in the detailed description of the embodiments. This summary is not intended to identify key or essential inventive concepts of the claimed subject matter, nor is it intended for determining the scope of the claimed subject matter.

As used herein, the phrase “medical/clinical” and variations on that phrase or the terms “medical” or “clinical” refer to the science of medicine or treatment of an illness and the research involving human test subjects.

According to some embodiments, a system to connect agents in a medical/clinical technology business-to-business marketplace is disclosed. The system includes at least one memory configured to store computer executable instructions; and at least one processor configured to execute the computer executable instructions to: receive first qualifying information from a first agent in the medical/clinical technology business-to-business marketplace; receive second qualifying information from a second agent in the medical/clinical technology business-to-business marketplace; determine a qualifying agent selected from one of the first agent or the second agent by filtering the first qualifying information or second qualifying information; and connect the qualifying agent with one of the first agent or the second agent.

According to some embodiments, a method for connecting agents in a medical/clinical technology business-to-business marketplace is disclosed. The method includes receiving first qualifying information from a first agent in the medical/clinical technology business-to-business marketplace; receiving second qualifying information from a second agent in the medical/clinical technology business-to-business marketplace; determining a qualifying agent selected from one of the first agent or the second agent by filtering the first qualifying information or second qualifying information; and connecting the qualifying agent with one of the first agent or the second agent.

According to some embodiments, a non-transitory computer readable medium is disclosed. The computer program product includes computer executable instructions stored thereon, which when executed by one or more processors, cause the one or more processors to connect agents in a medical/clinical technology business-to-business marketplace. The computer program product includes computer executable instructions to: receive first qualifying information from a first agent in the medical/clinical technology business-to-business marketplace; receive second qualifying information from a second agent in the medical/clinical technology business-to-business marketplace; determine a qualifying agent selected from one of the first agent or the second agent by filtering the first qualifying information or second qualifying information; and connect the qualifying agent with one of the first agent or the second agent.

Other systems, methods, features, and advantages of the disclosure will be, or will become, apparent to one with skill in the art upon examination of the following figures and detailed description. It is intended that all such additional systems, methods, features and advantages be included within this description, be within the scope of the disclosure, and be protected by the following claims.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1 shows components of one aspect of a computing environment for connecting agents in a medical/clinical technology business-to-business marketplace;

FIG. 2 shows one variation of a client computer for connecting agents in a medical/clinical technology business-to-business marketplace; and

FIG. 3 illustrates an exemplary block diagram of a system for connecting agents in a medical/clinical technology business-to-business marketplace.

The drawings are not necessarily to scale, and certain features and certain views of the drawings may be shown exaggerated in scale or in schematic in the interest of clarity and conciseness.

### DETAILED DESCRIPTION

The specific details of the single embodiment or variety of embodiments described herein are to the described system and methods of use. Any specific details of the embodiments are used for demonstration purposes only, and no unnecessary limitations or inferences are to be understood thereon.

Before describing in detail exemplary embodiments, it is noted that the embodiments reside primarily in combinations of components and procedures related to the system. Accordingly, the system components have been represented, where appropriate, by conventional symbols in the drawings, showing only those specific details that are pertinent to understanding the embodiments of the present disclosure so as not to obscure the disclosure with details that will be readily apparent to those of ordinary skill in the art having the benefit of the description herein.

In this disclosure, the various embodiments may be a system, method, or computer program product at any possible technical detail level of integration. A computer program product can include, among other things, a computer-readable storage medium having computer-readable program instructions thereon for causing a processor to carry out aspects of the present disclosure.

Some aspects of the present disclosure will now be described more fully hereinafter with reference to the accompanying drawings, in which some, but not all, aspects are shown. Indeed, various aspects may be embodied in many different forms and should not be construed as limited to the aspects set forth herein; rather, these aspects are provided so that this disclosure will satisfy applicable legal requirements. Like reference numerals refer to like elements throughout. As used herein, the terms “data,” “content,” “information,” and similar terms may be used interchangeably to refer to data capable of being transmitted, received or stored in accordance with aspects of the present disclosure. Thus, use of any such terms should not be made to limit the spirit and scope of aspects of the present disclosure.

The elements in the Figures interoperate as explained in more detail below. Before setting forth the detailed explanation, however, it is noted that all of the discussion below, regardless of the particular implementation being described, is exemplary in nature, rather than limiting. For example, although selected aspects, features, or components of the implementations are depicted as being stored in memories, all or part of systems and methods consistent with the display systems may be stored on, distributed across, or read from other machine-readable media, for example, secondary storage devices such as hard disks, floppy disks, and CD-ROMs; a signal received from a network; or other forms of ROM or RAM either currently known or later developed.

Furthermore, although specific components of the architecture will be described, methods, systems, and articles of manufacture consistent with the architecture may include additional or different components. For example, a processor may be implemented as a microprocessor, microcontroller, application specific integrated circuit (ASIC), discrete logic, or a combination of other type of circuits or logic. Similarly, memories, may be DRAM, SRAM, Flash, or any other type of memory. Flags, data, databases, tables, and other data structures may be separately stored and managed, may be incorporated into a single memory or database, may be distributed, or may be logically and physically organized in many different ways. Programs may be parts of a single program, separate programs, or distributed across several memories and processors.

In the following description, numerous specific details are set forth to clearly describe various specific aspects disclosed herein. One skilled in the art, however, will understand that the presently claimed disclosure may be practiced without all of the specific details discussed below. In other instances, well known features have not been described so as not to obscure the disclosure. As described herein, the term “pivotally connected” shall be used to describe a situation wherein two or more identified objects are joined together in a manner that allows one or both of the objects to pivot, or rotate about or in relation to the other object in either a horizontal or vertical manner. As described herein, the term “removably coupled” and derivatives thereof shall be used to describe a situation wherein two or more objects are joined together in a non-permanent manner so as to allow the same objects to be repeatedly joined and separated. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless limited otherwise, the terms “connected,” “coupled,” and “mounted,” and variations thereof herein are used broadly and encompass direct and indirect connections, couplings, and mountings. In addition, the terms “connected” and “coupled” and variations thereof are not restricted to physical or mechanical connections or couplings. In addition, it should be understood that aspects of the disclosure include both hardware and electronic components or modules that, for purposes of discussion, may be illustrated and described as if the majority of the components were implemented solely in hardware. However, one of ordinary skill in the art, and based on a reading of this detailed description, would recognize that, in at least one aspect, the electronic based aspects of the disclosure may be implemented in software. As such, it should be noted that a plurality of hardware and software-based devices, as well as a plurality of different structural components may be utilized to implement the disclosure. Furthermore, and as described in subsequent paragraphs, the specific mechanical configurations illustrated in the drawings are intended to exemplify aspects of the disclosure and that other alternative mechanical configurations are possible.

Currently, there is a lack of diversity in clinical research & development. For example, ethnic minorities and females experience higher rates of certain physical and mental illnesses but are typically under-represented in clinical studies. For cancer, African American (AA)/Black women (BW) have a higher rate of mortality, dying 41% more than white women from the same breast cancer diagnosis. However, BW represented only 3% of clinical trial participants for breast cancer drugs approved from 2008-2018. For infectious diseases, AA and Black people are 1.5 times more likely to get COVID-19. Latinx people are twice as likely to get COVID-19. People of color were underrepresented in late-stage COVID vaccine trials compared to their share of the population. Additionally, for dementia, AA/Black people are two times more likely than white people to develop Alzheimer's disease (AD) yet represent only 2% of those in clinical trials. Hispanics are about one and one-half times more likely than whites to have AD and other dementias.

Despite people of color being more likely to contract COVID-19, become hospitalized and die, communities of color resisted receiving the COVID-19 vaccination. The United States government and nations alike attempted to build trust among communities during the plight of COVID-19 but resistance persisted. Subtly addressing healthcare mistrust among people of color during a pandemic is not enough to sustain diversity and inclusion in clinical research and development.

Biopharmaceutical, Biotechnology, Medical device, and other companies alike, lack diverse employment practices and minority representation within their companies. For example, 12% of Biopharmaceutical and Biotechnology companies within a certain region employ no people of color. More importantly, Executive and board level positions within these companies do not reflect the populations in which these companies are marketing or selling products to. Therefore, the clinical decision making, impacting the masses, comes into public questioning.

The clinical research & development conducted by these companies are still perceived in the same light as the Tuskegee Syphilis experiment-white researchers experimenting on people of color whilst violating bioethical principles of respect for autonomy. This perception among certain populations will continue until the inside of organizations and engaged service providers reflect the necessary diversity within clinical research & development. Diversifying marketing materials will never be enough and does not address the root cause.

Lack of diversity in research persists because of a vicious product development lifecycle and governmental difficulty in holding Sponsors (e.g., Biopharmaceutical and Biotechnology companies) accountable for research diversity while also putting the public first: (1) Sponsors conduct research within a non-diverse population submitting applications for marketing authorization, (2) Health authorities (HA) (e.g., Food and Drug Administration, Health Canada, European Medicines Agency) push back questioning generalizability of study data due to lack of diversity among study participants, (3) Sponsors respond to HA concerns promising to pursue diversity in future research initiatives pleading for approval for patients who need options now, and finally, (4) The HA approves the Sponsor's product in efforts to meet the needs of the public. Then, the cycle repeats.

Another problem relates to delayed research initiatives due to difficulty finding talent and relevant Service Providers (Suppliers). Big, medium-sized, and small Biopharmaceutical and Biotechnology companies contract external recruiting firms to discover talent in various departments (e.g., clinical affairs, medical affairs, regulatory affairs). Many of these vendors resort to spamming techniques which results in people thinking many jobs in clinical research are a “scam.”

The current talent pool within clinical research is limited. There are more jobs than there is talent to fill them. Limited talent makes it a challenge for Sponsors to execute research initiatives and efficiently provide treatment options for patients. Diverse and qualified talent (e.g., BS, MPH, RN's, PharmD's) are also unaware of opportunities within clinical research due to limited knowledge about the industry or skepticism.

Sponsors often use Contract Research Organizations (CROs) or consulting firms to execute various research tasks (e.g., data management, trial monitoring, regulatory affairs consulting, pharmacovigilance). The smaller the organization the more dependence there is on such Suppliers. Sponsors spend months searching for and qualifying suppliers to support their research. Suppliers with relevant therapeutic experience is essential. Removing the time to discover qualified suppliers would expedite research initiatives tremendously.

Therefore, a need exists for a B2B software platform to increase diversity among the clinical research workforce and trust within diverse communities. According to some embodiments, the B2B software platform connects Biopharmaceutical and Biotechnology companies with a pool of qualified clinical research suppliers. In another aspect, the B2B software platform connects a diverse pool of qualified talent/professionals with Biopharmaceutical and Biotechnology companies. In another aspect, the B2B software platform cuts costs for talent recruitment and supplier discovery for Biopharmaceutical and Biotechnology companies. In another aspect, the B2B software platform connects suppliers with more opportunities.

According to some aspects, the presently disclosed B2B system or method will allow users to discover diverse suppliers by filtering by therapeutic relevance, by filtering by supplier type (e.g., regulatory consulting, CROs, etc.), and allow sponsors to submit inquires and a supplier to provide a response utilizing inboxes implemented by the B2B software platform.

According to some aspects of the disclosure, the presently disclosed B2B system or method allows users to discover diverse talent by viewing talent profiles, filtering by education, filtering by years of experience, communicating with talent and scheduling interviews utilizing inboxes implemented by the B2B software platform.

According to some embodiments, a system to connect agents in a medical/clinical technology business-to-business marketplace is disclosed. The system is configured to receive first qualifying information from a first agent in the medical/clinical technology business-to-business marketplace; receive second qualifying information from a second agent in the medical/clinical technology business-to-business marketplace; determine a qualifying agent selected from one of the first agent or the second agent by filtering the first qualifying information or second qualifying information; and connect the qualifying agent with one of the first agent or the second agent.

In an aspect of the disclosed B2B system, an agent may be a medical/clinical technology research company in the B2B marketplace, a medical/clinical technology supplier in the B2B marketplace or a medical/clinical technology employee candidate. It is understood that “medical technology” may be biotechnology (“biotech”), biopharmaceutical technology (“biopharma”), medical/clinical instrument technology, medical/clinical electronics technology, medical/clinical detection equipment or other medical/clinical technology that typically utilizes clinical research and technology to develop and promote products or services related to the medical/clinical technology B2B marketplace.

The following briefly describes the aspects of the disclosure in order to provide a basic understanding of some aspects of the disclosure. This brief description is not intended as an extensive overview. It is not intended to identify key or critical elements, or to delineate or otherwise narrow the scope. Its purpose is merely to present some concepts in a simplified form as a prelude to the more detailed description that is presented later.

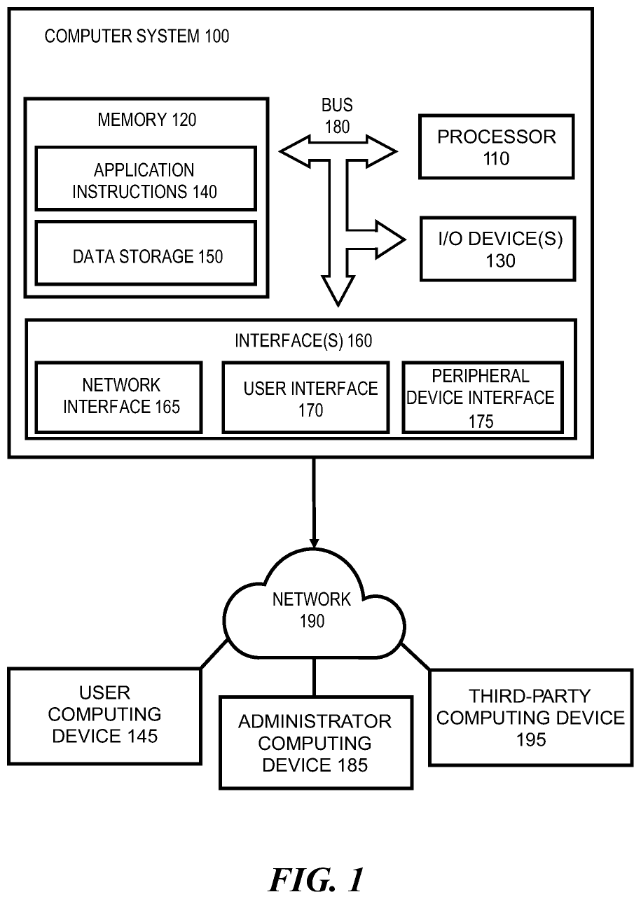

FIG. 1 illustrates an example of a computer system 100 that may be utilized to execute various procedures, including the processes described herein. Computer system 100 comprises a standalone computer or mobile computing device, a mainframe computer system, a workstation, a network computer, a desktop computer, a laptop, or the like. The computing device 100 can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive).

In some embodiments, the computer system 100 includes one or more processors 110 coupled to a memory 120 through a system bus 180 that couples various system components, such as an input/output (I/O) devices 130, to the processors 110. The bus 180 may be any of several types of bus structures including a memory bus or memory controller, a peripheral bus, and a local bus using any of a variety of bus architectures. For example, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, also known as Mezzanine bus.

In some embodiments, the computer system 100 includes one or more input/output (I/O) devices 130, such as video device(s) (e.g., a camera), audio device(s), and display(s) are in operable communication with the computer system 100. In some embodiments, similar I/O devices 130 may be separate from the computer system 100 and may interact with one or more nodes of the computer system 100 through a wired or wireless connection, such as over a network interface.

Processors 110 suitable for the execution of computer readable program instructions include both general and special purpose microprocessors and any one or more processors of any digital computing device. For example, each processor 110 may be a single processing unit or a number of processing units and may include single or multiple computing units or multiple processing cores. The processor(s) 110 can be implemented as one or more microprocessors, microcomputers, microcontrollers, digital signal processors, central processing units, state machines, logic circuitries, or any devices that manipulate signals based on operational instructions. For example, the processor(s) 110 may be one or more hardware processors or logic circuits of any suitable type specifically programmed or configured to execute the algorithms and processes described herein. The processor(s) 110 can be configured to fetch and execute computer readable program instructions stored in the computer-readable media, which can program the processor(s) 110 to perform the functions described herein.

In this disclosure, the term “processor” can refer to substantially any computing processing unit or device, including single-core processors, single-processors with software multithreading execution capability, multi-core processors, multi-core processors with software multithreading execution capability, multi-core processors with hardware multithread technology, parallel platforms, and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures, such as molecular and quantum-dot based transistors, switches, and gates, to optimize space usage or enhance performance of user equipment. A processor can also be implemented as a combination of computing processing units.

In some embodiments, the memory 120 includes computer-readable application instructions 150, configured to implement certain embodiments described herein, and a database 150, comprising various data accessible by the application instructions 140. In some embodiments, the application instructions 140 include software elements corresponding to one or more of the various embodiments described herein. For example, application instructions 140 may be implemented in various embodiments using any desired programming language, scripting language, or combination of programming or scripting languages (e.g., C, C++, C#, JAVA, JAVASCRIPT, PERL, etc.).

In this disclosure, terms “store,” “storage,” “data store,” data storage,” “database,” and substantially any other information storage component relevant to operation and functionality of a component are utilized to refer to “memory components,” which are entities embodied in a “memory,” or components comprising a memory. Those skilled in the art would appreciate that the memory or memory components described herein can be volatile memory, nonvolatile memory, or both volatile and nonvolatile memory. Nonvolatile memory can include, for example, read only memory (ROM), programmable ROM (PROM), electrically programmable ROM (EPROM), electrically erasable ROM (EEPROM), flash memory, or nonvolatile random-access memory (RAM) (e.g., ferroelectric RAM (FeRAM). Volatile memory can include, for example, RAM, which can act as external cache memory. The memory or memory components of the systems or computer-implemented methods can include the foregoing or other suitable types of memory.

Generally, a computing device will also include, or be operatively coupled to receive data from or transfer data to, or both, one or more mass data storage devices; however, a computing device need not have such devices. The computer readable storage medium (or media) can be a tangible device that can retain and store instructions for use by an instruction execution device. The computer readable storage medium can be, for example, an electronic storage device, a magnetic storage device, an optical storage device, an electromagnetic storage device, a semiconductor storage device, or any suitable combination of the foregoing. A non-exhaustive list of more specific examples of the computer readable storage medium can include: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. In this disclosure, a computer readable storage medium is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

In some embodiments, the steps and actions of the application instructions 140 described herein are embodied directly in hardware, in a software module executed by a processor, or in a combination of the two. A software module may reside in RAM, flash memory, ROM memory, EPROM memory, EEPROM memory, registers, a hard disk, a removable disk, a CD-ROM, or any other form of storage medium known in the art. An exemplary storage medium may be coupled to the processor 110 such that the processor 110 can read information from, and write information to, the storage medium. In the alternative, the storage medium may be integrated into the processor 110. Further, in some embodiments, the processor 110 and the storage medium may reside in an Application Specific Integrated Circuit (ASIC). In the alternative, the processor and the storage medium may reside as discrete components in a computing device. Additionally, in some embodiments, the events or actions of a method or algorithm may reside as one or any combination or set of codes and instructions on a machine-readable medium or computer-readable medium, which may be incorporated into a computer program product.

In some embodiments, the application instructions 140 for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The application instructions 140 can execute entirely on the user's computer, partly on the user's computer, as a stand-alone software package, partly on the user's computer and partly on a remote computer, or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

In some embodiments, the application instructions 140 can be downloaded to a computing/processing device from a computer readable storage medium, or to an external computer or external storage device via a network 190. A network adapter card or network interface in each computing/processing device receives computer readable program instructions from the network and forwards the computer readable application instructions 140 for storage in a computer readable storage medium within the respective computing/processing device.

In some embodiments, the computer system 100 includes one or more interfaces 160 that allow the computer system 100 to interact with other systems, devices, or computing environments. In some embodiments, the computer system 100 comprises a network interface 165 to communicate with a network 190. In some embodiments, the network interface 165 is configured to allow data to be exchanged between the computer system 100 and other devices attached to the network 190, such as other computer systems, or between nodes of the computer system 100. In various embodiments, the network interface 165 may support communication via wired or wireless general data networks, such as any suitable type of Ethernet network, for example, via telecommunications/telephony networks such as analog voice networks or digital fiber communications networks, via storage area networks such as Fiber Channel SANs, or via any other suitable type of network or protocol. Other interfaces include the user interface 170 and the peripheral device interface 175.

In some embodiments, the network 190 corresponds to a local area network (LAN), wide area network (WAN), the Internet, a direct peer-to-peer network (e.g., device to device Wi-Fi, Bluetooth, etc.), or an indirect peer-to-peer network (e.g., devices communicating through a server, router, or other network device). The network 190 can comprise copper transmission cables, optical transmission fibers, wireless transmission, routers, firewalls, switches, gateway computers or edge servers. The network 190 can represent a single network or multiple networks. In some embodiments, the network 190 used by the various devices of the computer system 100 is selected based on the proximity of the devices to one another or some other factor. For example, when a first user device and second user device are near each other (e.g., within a threshold distance, within direct communication range, etc.), the first user device may exchange data using a direct peer-to-peer network. But when the first user device and the second user device are not near each other, the first user device and the second user device may exchange data using a peer-to-peer network (e.g., the Internet). The Internet refers to the specific collection of networks and routers communicating using an Internet Protocol (“IP”) including higher level protocols, such as Transmission Control Protocol/Internet Protocol (“TCP/IP”) or the Uniform Datagram Packet/Internet Protocol (“UDP/IP”).

Any connection between the components of the system may be associated with a computer-readable medium. For example, if software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. As used herein, the terms “disk” and “disc” include compact disc (CD), laser disc, optical disc, digital versatile disc (DVD), floppy disk, and Blu-ray disc; in which “disks” usually reproduce data magnetically, and “discs” usually reproduce data optically with lasers. Combinations of the above should also be included within the scope of computer-readable media. In some embodiments, the computer-readable media includes volatile and nonvolatile memory or removable and non-removable media implemented in any type of technology for storage of information, such as computer-readable instructions, data structures, program modules, or other data. Such computer-readable media may include RAM, ROM, EEPROM, flash memory or other memory technology, optical storage, solid state storage, magnetic tape, magnetic disk storage, RAID storage systems, storage arrays, network attached storage, storage area networks, cloud storage, or any other medium that can be used to store the desired information and that can be accessed by a computing device. Depending on the configuration of the computing device, the computer-readable media may be a type of computer-readable storage media or a tangible non-transitory media to the extent that when mentioned, non-transitory computer-readable media exclude media such as energy, carrier signals, electromagnetic waves, and signals per se.

In some embodiments, the system is world-wide-web (www) based, and the network server is a web server delivering HTML, XML, etc., web pages to the computing devices. In other embodiments, a client-server architecture may be implemented, in which a network server executes enterprise and custom software, exchanging data with custom client applications running on the computing device.

In some embodiments, the system can also be implemented in cloud computing environments. In this context, “cloud computing” refers to a model for enabling ubiquitous, convenient, on-demand network access to a shared pool of configurable computing resources (e.g., networks, servers, storage, applications, and services) that can be rapidly provisioned via virtualization and released with minimal management effort or service provider interaction, and then scaled accordingly. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

As used herein, the term “add-on” (or “plug-in”) refers to computing instructions configured to extend the functionality of a computer program, where the add-on is developed specifically for the computer program. The term “add-on data” refers to data included with, generated by, or organized by an add-on. Computer programs can include computing instructions, or an application programming interface (API) configured for communication between the computer program and an add-on. For example, a computer program can be configured to look in a specific directory for add-ons developed for the specific computer program. To add an add-on to a computer program, for example, a user can download the add-on from a website and install the add-on in an appropriate directory on the user's computer.

In some embodiments, the computer system 100 may include a user computing device 145, an administrator computing device 185 and a third-party computing device 195 each in communication via the network 190. The user computing device 145 may be utilized to establish credentials, create a user profile, and otherwise interact with the various property management and real estate investment functionalities of the system. The third-party computing device 195 may be utilized by third parties to receive communications from the user computing device or administrative computing device 185.

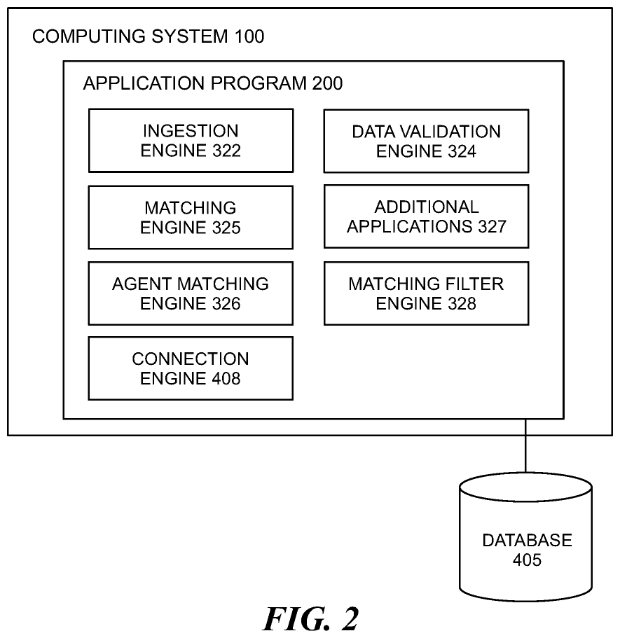

FIG. 2 depicts a computing system 100 implementing an application program 200 including an ingestion engine 322, a data validation engine 324, matching engine 325, or other applications 327. In at least one of the various aspects, one or more of the applications may be implemented as modules or components of another application. Further, in at least one of the various aspects, applications may be implemented as operating system extensions, modules, plugins, or the like.

Furthermore, in at least one of the various aspects, the ingestion engine 322 for receiving agent information related to the disclosed medical technology B2B marketplace, the data validation engine 324 for confirming data integrity and applicability for processing by the system, the matching engine 325 for determining matches between agents in the medical technology B2B marketplace, or other applications 326, or the like, may be operative in a cloud-based computing environment. In at least one of the various aspects, these applications, and others, which comprise the management platform may be executing within virtual machines or virtual servers that may be managed in a cloud-based based computing environment. In at least one of the various aspects, in this context the applications may flow from one physical network computer within the cloud-based environment to another depending on performance and scaling considerations automatically managed by the cloud computing environment. Likewise, in at least one of the various aspects, virtual machines or virtual servers dedicated to the ingestion engine 322, the data validation engine 324, the matching engine 325, or the other applications 326, may be provisioned and de-commissioned automatically.

In at least one of the various aspects, the applications may be arranged to employ geo-location information to select one or more localization features, such as, time zones, languages, currencies, calendar formatting, or the like. Localization features may be used in user-interfaces as well as internal processes or databases. In at least one of the various aspects, geo-location information used for selecting localization information may be provided by the GPS transceiver. Also, in some aspects, geolocation information may include information providing using one or more geolocation protocol over the networks, such as, the wireless network or the network.

Also, in at least one of the various aspects, the ingestion engine 322, the data validation engine 324, the matching engine 325, or the other applications 326, or the like, may be located in virtual servers running in a cloud-based computing environment rather than being tied to one or more specific physical network computers.

The system includes an agent information input system or ingestion engine 322, where an agent's qualifying information that the agent wishes to search against, is provided to the system. As described herein, an agent is a person or entity in the medical technology field, such as, According to some embodiments, clinical testing for medical products or services, who wishes to use the disclosed medical technology B2B system to connect with other agents in the B2B marketplace, to find qualified medical technology research companies, medical technology suppliers or medical technology employee candidates. An agent may be one of several agents in B2B marketplace, such as a first agent, a second agent or more. An agent may enter, into the ingestion engine 322, qualifying information such as a medical technology area, an employment opportunity, an employment opportunity experience level, an employment opportunity education level, an employee candidate education level, an employee candidate experience level, geographic information for the first agent or the second agent, diversity information about the first agent or the second agent, a supplier technology field, a supplier type, a supplier therapeutic relevance, etc. The agent qualifying information is transferred to the agent matching engine 326 for further processing and output of a match between a first agent and at least a second agent or other agents. At data validation engine 324, the system processes the agent qualifying information to confirm data quality, conformity with standards for input and processing, applicability of the agent's qualifying information for processing in the disclosed medical technology B2B marketplace system or other data validation steps needed for further processing by the system 100, the system may select sampling/resampling options for processing the audio, either through pre-selected options or user-selected options.

A matching filter engine 328 may be used to determine a qualifying agent selected from one of the first agent or the second agent by filtering the first qualifying information of the first agent or the second qualifying information from the second agent. The matching filter engine 328 may compare subsets of the qualifying information or the entire set of qualifying information from the agents in the system. According to some embodiments, the matching filter engine 328 may match medical technology company with a qualifying medical technology employee candidate or to match the medical technology company with a qualifying medical technology supplier. The medical technology company may seek out medical technology suppliers that satisfy certain qualifying information requirements, such as the therapeutic relevance of the supplier, supplier type (i.e., regulatory consulting, CROs, etc.), reputation, diversity measures or diversity information about the medical technology supplier, etc. The medical technology company may seek out talent for open employment positions within the medical technology company, by evaluating qualifying information of the medical technology employee candidate, such as geographic location, education level, experience level and type of experience, demographic and diversity information, previous company information, etc. Likewise, the matching filter engine 328 may allow a medical technology employee candidate to evaluate medical technology companies using similar qualifying information, such as open employment or internship opportunities, education level and experience associated with open opportunities within the company, geographic information, diversity information about the medical technology company, reputation, corporate governance information, social, ethical and environmental responsibility, etc.

The matching filter engine 328 may determine a qualifying agent from the at least first agent and second agent by matching the qualifying candidate with the first agent or the second agent based on a highest match level of the first qualifying information and the second qualifying information. The matching filter engine 328 may apply a weighting to certain qualifying information based on agent input, past agent selections or actions within the system, weights derived from modeling data in the system or modeled as a machine learning model to predict a qualifying agent based on qualifying information supplied by agents or other data samples.

A database 405 may be operatively connected to the agent mapping engine 402. The database 405 may be contained within the agent mapping engine 402, located remotely from the agent mapping engine 402 or may be a distributed database across a network and connected devices. The database 405 may store agent qualifying information, software code for the matching filter engine 328 and other engines within the agent mapping engine 402.

The agent matching engine 325 may include a matching engine 325. The matching engine 325 may confirm the quality of the match between an agent and a corresponding qualifying agent. The matching engine 325 may examine whether the qualifying agent is still available in the database 405, whether match is the highest match based on previous operations by the agent mapping engine 402, or other output quality determinations.

Once a match is determined for a qualifying agent to a first agent, second agent, or more agents, the system will employ an agent connection engine 408. The agent connection engine 408 may connect an agent with its corresponding qualifying agent(s) to facilitate communication between the agents. According to some embodiments, the agent connection engine 408 may provide a query inbox for the qualifying agent and the one of the first agent or the second agent to exchange information or queries related to the medical technology business-to-business marketplace. According to some embodiments, the query inbox could be an email inbox on an email server, web template for an agent to fill in to connect with a qualifying agent, a video “mailbox”, an SMS or other text message interface that allows agents to connect, or other mechanisms known to one of skill in the art to facilitation connection between agents in the medical technology B2B marketplace.

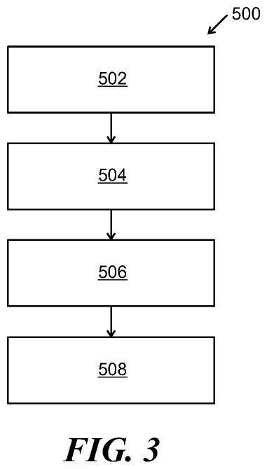

FIG. 5 illustrates a flowchart 500 for acts taken in an exemplary method for connecting agents in a medical technology B2B marketplace. FIG. 5 is explained in conjunction with FIG. 1 to FIG. 4. The control starts at act 502.

At act 502, the system receives or ingests receive first qualifying information from a first agent. The first agent may be a medical technology research company or a prospective medical technology employee candidate. The first agent qualifying information may be a medical technology area, an employment opportunity, an employment opportunity experience level, an employment opportunity education level, an employee candidate education level, an employee candidate experience level, geographic information for the first agent or the second agent, diversity information about the first agent or the second agent, a supplier technology field, a supplier type, a supplier therapeutic relevance or a combination thereof.

At act 504, the system receives or ingests receive second qualifying information from a second agent. The second agent may be a medical technology research company, a medical technology supplier or a medical technology employee candidate. The second agent qualifying information may be a medical technology area, an employment opportunity, an employment opportunity experience level, an employment opportunity education level, an employee candidate education level, an employee candidate experience level, geographic information for the first agent or the second agent, diversity information about the first agent or the second agent, a supplier technology field, a supplier type, a supplier therapeutic relevance or a combination thereof.

At act 506, the system may determine a qualifying agent selected from one of the first agent or the second agent by filtering the first qualifying information or second qualifying information. According to some embodiments, the system may determine the qualifying agent by matching the medical technology company with a qualifying medical technology employee candidate or by match the medical technology company with a qualifying medical technology supplier.

According to some embodiments, disclosed medical technology B2B marketplace software platform may match medical technology company with a qualifying medical technology employee candidate or to match the medical technology company with a qualifying medical technology supplier. The medical technology company may seek out medical technology suppliers that satisfy certain qualifying information requirements, such as the therapeutic relevance of the supplier, supplier type (i.e., regulatory consulting, CROs, etc.), reputation, diversity measures or diversity information about the medical technology supplier, etc. The medical technology company may seek out talent for open employment positions within the medical technology company, by evaluating qualifying information of the medical technology employee candidate, such as geographic location, education level, experience level and type of experience, demographic and diversity information, previous company information, etc.

According to some embodiments, the system may determine the qualifying agent by matching a medical technology employee candidate with prospective medical technology companies that the medical technology employee candidate finds attractive based on the qualifying information of the medical technology company. According to some embodiments, and as described herein, the disclosed B2B marketplace software platform may allow a medical technology employee candidate to evaluate medical technology companies using similar qualifying information, such as open employment or internship opportunities, education level and experience associated with open opportunities within the company, geographic information, diversity information about the medical technology company, reputation, corporate governance information, social, ethical and environmental responsibility, etc.

According to some embodiments, the system may determine a qualifying agent from the at least first agent and second agent by matching the qualifying candidate with the first agent or the second agent based on a highest match level of the first qualifying information and the second qualifying information. The matching filter engine 328 may apply a weighting to certain qualifying information based on agent input, past agent selections or actions within the system, weights derived from modeling data in the system or modeled as a machine learning model to predict a qualifying agent based on qualifying information supplied by agents or other data samples.

At act 508, the system connects the qualifying agent with the agent that matches with the qualifying agent based on qualifying information provided by the agent and agent preferences. The system may connect an agent with its corresponding qualifying agent(s) to facilitate communication between the agents. According to some embodiments, the system may provide a query inbox for the qualifying agent and the one of the first agent or the second agent to exchange information or queries related to the medical technology business-to-business marketplace. According to some embodiments, the query inbox could be an email inbox on an email server, web template for an agent to fill in to connect with a qualifying agent, a video “mailbox”, an SMS or other text message interface that allows agents to connect, or other mechanisms known to one of skill in the art to facilitation connection between agents in the medical technology B2B marketplace.

Users of the disclosed system, such as the agents described herein, would utilize the B2B marketplace to increase diversity among the clinical research & development workforce and trust within diverse communities, resulting to diverse clinical trials, by connecting with medical technology companies, medical technology suppliers and medical technology employee candidates using the software. Connecting among all three users would cut talent and supplier recruitment costs for medical technology companies and medical supplier companies and increase the awareness of opportunities in clinical research among diverse talent. Connection among all three users would improve community awareness and greater visibility into clinical research initiatives resulting in a greater trust in the research being performed.

Blocks of the flowchart 500 support combinations of means for performing the specified functions and combinations of operations for performing the specified functions. It will also be understood that one or more blocks of the flowchart 500, and combinations of blocks in the flowchart 500, can be implemented by special purpose hardware-based computer systems which perform the specified functions, or combinations of special purpose hardware and computer instructions. Also, more, fewer or different steps may be provided.

Alternatively, the system may comprise means for performing each of the operations described above. In this regard, according to an example aspect, examples of means for performing operations may comprise, for example, the processor or a device or circuit for executing instructions or executing an algorithm for processing information as described above.

It will be understood that each block of the flowcharts and combination of blocks in the flowcharts may be implemented by various means, such as hardware, firmware, processor, circuitry, or other communication devices associated with execution of software including one or more computer program instructions. For example, one or more of the procedures described above may be embodied by computer program instructions. In this regard, the computer program instructions which embody the procedures described above may be stored by a memory device of an apparatus employing an aspect of the present disclosure and executed by the processing circuitry. As will be appreciated, any such computer program instructions may be loaded onto a computer or other programmable apparatus (for example, hardware) to produce a machine, such that the resulting computer or other programmable apparatus implements the functions specified in the flowchart blocks. These computer program instructions may also be stored in a computer-readable memory that may direct a computer or other programmable apparatus to function in a particular manner, such that the instructions stored in the computer-readable memory produce an article of manufacture the execution of which implements the function specified in the flowchart blocks. The computer program instructions may also be loaded onto a computer or other programmable apparatus to cause a series of operations to be performed on the computer or other programmable apparatus to produce a computer-implemented process such that the instructions which execute on the computer or other programmable apparatus provide operations for implementing the functions specified in the flowchart blocks.

A “computer-readable medium,” “machine-readable medium,” “propagated-signal” medium, or “signal-bearing medium” may comprise any means that contains, stores, communicates, propagates, or transports software for use by or in connection with an instruction executable system, apparatus, or device. The machine-readable medium may selectively be, but not limited to, an electronic, magnetic, optical, electromagnetic, infrared, or semiconductor system, apparatus, device, or propagation medium. A non-exhaustive list of examples of a machine-readable medium would include: an electrical connection “electronic” having one or more wires, a portable magnetic or optical disk, a volatile memory such as a Random Access Memory “RAM” (electronic), a Read-Only Memory “ROM” (electronic), an Erasable Programmable Read-Only Memory (EPROM or Flash memory) (electronic), or an optical fiber (optical). A machine-readable medium may also include a tangible medium upon which software is printed, as the software may be electronically stored as an image or in another format (e.g., through an optical scan), then compiled, or interpreted or otherwise processed. The processed medium may then be stored in a computer or machine memory.

The following description of variants is only illustrative of components, elements, acts, product, and methods considered to be within the scope of the invention and are not in any way intended to limit such scope by what is specifically disclosed or not expressly set forth. The components, elements, acts, product, and methods as described herein may be combined and rearranged other than as expressly described herein and still are considered to be within the scope of the invention.

According to variation 1, a system to connect agents in a medical/clinical technology business-to-business marketplace may include at least one memory configured to store computer executable instructions; and at least one processor configured to execute the computer executable instructions to receive first qualifying information from a first agent in the medical/clinical technology business-to-business marketplace; receive second qualifying information from a second agent in the medical/clinical technology business-to-business marketplace; determine a qualifying agent selected from one of the first agent or the second agent by filtering the first qualifying information or second qualifying information; and connect the qualifying agent with one of the first agent or the second agent.

Variation 2 may include a system as in variation 1, where the first qualifying information or the second qualifying information includes a medical/clinical technology area, an employment opportunity, an employment opportunity experience level, an employment opportunity education level, an employee candidate education level, an employee candidate experience level, geographic information for the first agent or the second agent, diversity information about the first agent or the second agent, a supplier technology field, a supplier type, a supplier therapeutic relevance or a combination thereof.

Variation 3 may include a system as in variation 1 or 2, where the first agent includes a medical/clinical technology research company or a prospective medical/clinical technology employee candidate.

Variation 4 may include a system as in any of variations 1 through 3, where the second agent includes a medical/clinical technology research company, a medical technology supplier or a medical/clinical technology employee candidate.

Variation 5 may include a system as in any of variations 1 through 4, where the computer executable instructions to determine the qualifying agent include computer executable instructions to match the medical/clinical technology company with a qualifying medical/clinical technology employee candidate or to match the medical/clinical technology company with a qualifying medical/clinical technology supplier.

Variation 6 may include a system as in any of variations 1 through 5, where the computer executable instructions to determine the qualifying agent include the computer executable instructions to match the qualifying candidate with the first agent or the second agent based on a highest match level of the first qualifying information and the second qualifying information.

Variation 7 may include a system as in any of variations 1 through 6, where the computer executable instructions to connect the qualifying agent with one of the first agent or the second agent include the computer executable instructions to provide a query inbox for the qualifying agent and the one of the first agent or the second agent to exchange information or queries related to the medical/clinical technology business-to-business marketplace.

According to variation 8, a method to connect agents in a medical/clinical technology business-to-business marketplace may include receiving first qualifying information from a first agent in the medical/clinical technology business-to-business marketplace; receiving second qualifying information from a second agent in the medical/clinical technology business-to-business marketplace; determining a qualifying agent selected from one of the first agent or the second agent by filtering the first qualifying information or second qualifying information; and connecting the qualifying agent with one of the first agent or the second agent.

Variation 9 may include a method as in variation 8, where the first qualifying information or the second qualifying information includes a medical/clinical technology area, an employment opportunity, an employment opportunity experience level, an employment opportunity education level, an employee candidate education level, an employee candidate experience level, geographic information for the first agent or the second agent, diversity information about the first agent or the second agent, a supplier technology field, a supplier type, a supplier therapeutic relevance or a combination thereof.

Variation 10 may include a method as in variation 8 or 9, where the second agent includes a medical/clinical technology research company, a medical/clinical technology supplier or a medical/clinical technology employee candidate.

Variation 11 may include a method as in any of variations 8 through 10, where determining the qualifying agent includes matching the medical/clinical technology company with a qualifying medical/clinical technology employee candidate or matching the medical/clinical technology company with a qualifying medical/clinical technology supplier.

Variation 12 may include a method as in any of variations 8 through 11, where determining the qualifying agent includes matching the qualifying candidate with the first agent or the second agent based on a highest match level of the first qualifying information and the second qualifying information.

Variation 13 may include a method as in any of variations 8 through 12, where connecting the qualifying agent with one of the first agent or the second agent includes providing a query inbox for the qualifying agent and the one of the first agent or the second agent to exchange information or queries related to the medical/clinical technology business-to-business marketplace.

According to variation 14, a computer program product including a non-transitory computer readable medium having stored thereon computer executable instructions, which when executed by one or more processors, cause the one or more processors to connect agents in a medical/clinical technology business-to-business marketplace, the operations including receiving first qualifying information from a first agent in the medical/clinical technology business-to-business marketplace; receiving second qualifying information from a second agent in the medical/clinical technology business-to-business marketplace; determining a qualifying agent selected from one of the first agent or the second agent by filtering the first qualifying information or second qualifying information; and connecting the qualifying agent with one of the first agent or the second agent.

Variation 15 may include a computer program product as in variation 14, where the first qualifying information or the second qualifying information includes a medical/clinical technology area, an employment opportunity, an employment opportunity experience level, an employment opportunity education level, an employee candidate education level, an employee candidate experience level, geographic information for the first agent or the second agent, diversity information about the first agent or the second agent, a supplier technology field, a supplier type, a supplier therapeutic relevance or a combination thereof.

Variation 16 may include a computer program product as in variation 14 or 15, where the first agent includes a medical/clinical technology research company or a prospective medical/clinical technology employee candidate.

Variation 17 may include a computer program product as in any of variations 14 through 16, where the second agent includes a medical/clinical technology research company, a medical/clinical technology supplier or a medical/clinical technology employee candidate.

Variation 18 may include a computer program product as in any of variations 14 through 17, where the computer executable instructions to determine the qualifying agent include computer executable instructions to match the medical/clinical technology company with a qualifying medical/clinical technology employee candidate or to match the medical/clinical technology company with a qualifying medical/clinical technology supplier.

Variation 19 may include a computer program product as in any of variations 14 through 18, where the computer executable instructions to determine the qualifying agent include computer executable instructions to match the qualifying candidate with the first agent or the second agent based on a highest match level of the first qualifying information and the second qualifying information.

Variation 20 may include a computer program product as in any of variations 14 through 19, where the computer executable instructions to connect the qualifying agent with one of the first agent or the second agent include computer executable instructions to provide a query inbox for the qualifying agent and the one of the first agent or the second agent to exchange information or queries related to the medical/clinical technology business-to-business marketplace.

In this disclosure, the descriptions of the various embodiments have been presented for purposes of illustration and are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein. Thus, the appended claims should be construed broadly, to include other variants and embodiments, which may be made by those skilled in the art.

What is claimed is:

1. A system to connect agents in a medical/clinical technology business-to-business marketplace, the system comprising:
at least one memory configured to store computer executable instructions; and
at least one processor configured to execute the computer executable instructions to:
receive first qualifying information from a first agent in the medical/clinical technology business-to-business marketplace;
receive second qualifying information from a second agent in the medical/clinical technology business-to-business marketplace;
determine a qualifying agent selected from one of the first agent or the second agent by filtering the first qualifying information or second qualifying information; and
connect the qualifying agent with one of the first agent or the second agent.

2. The system of claim 1, where the first qualifying information or the second qualifying information comprises a medical/clinical technology area, an employment opportunity, an employment opportunity experience level, an employment opportunity education level, an employee candidate education level, an employee candidate experience level, geographic information for the first agent or the second agent, diversity information about the first agent or the second agent, a supplier technology field, a supplier type, a supplier therapeutic relevance or a combination thereof.

3. The system of claim 1, where the first agent comprises a medical/clinical technology research company or a prospective medical/clinical technology employee candidate.

4. The system of claim 3, where the second agent comprises a medical/clinical technology research company, a medical technology supplier or a medical/clinical technology employee candidate.

5. The system of claim 4, where the computer executable instructions to determine the qualifying agent comprise computer executable instructions to match the medical/clinical technology company with a qualifying medical/clinical technology employee candidate or to match the medical/clinical technology company with a qualifying medical/clinical technology supplier.

6. The system of claim 1, where the computer executable instructions to determine the qualifying agent comprise the computer executable instructions to match the qualifying candidate with the first agent or the second agent based on a highest match level of the first qualifying information and the second qualifying information.

7. The system of claim 1, where the computer executable instructions to connect the qualifying agent with one of the first agent or the second agent comprise the computer executable instructions to provide a query inbox for the qualifying agent and the one of the first agent or the second agent to exchange information or queries related to the medical/clinical technology business-to-business marketplace.

8. A method to connect agents in a medical/clinical technology business-to-business marketplace, the method comprising:
receiving first qualifying information from a first agent in the medical/clinical technology business-to-business marketplace;
receiving second qualifying information from a second agent in the medical/clinical technology business-to-business marketplace;
determining a qualifying agent selected from one of the first agent or the second agent by filtering the first qualifying information or second qualifying information; and
connecting the qualifying agent with one of the first agent or the second agent.

9. The method of claim 8, where the first qualifying information or the second qualifying information comprises a medical/clinical technology area, an employment opportunity, an employment opportunity experience level, an employment opportunity education level, an employee candidate education level, an employee candidate experience level, geographic information for the first agent or the second agent, diversity information about the first agent or the second agent, a supplier technology field, a supplier type, a supplier therapeutic relevance or a combination thereof.

10. The method of claim 9, where the second agent comprises a medical/clinical technology research company, a medical/clinical technology supplier or a medical/clinical technology employee candidate.

11. The method of claim 10, where determining the qualifying agent comprises matching the medical/clinical technology company with a qualifying medical/clinical technology employee candidate or matching the medical/clinical technology company with a qualifying medical/clinical technology supplier.

12. The method of claim 8, where determining the qualifying agent comprises matching the qualifying candidate with the first agent or the second agent based on a highest match level of the first qualifying information and the second qualifying information.

13. The method of claim 8, where connecting the qualifying agent with one of the first agent or the second agent comprises providing a query inbox for the qualifying agent and the one of the first agent or the second agent to exchange information or queries related to the medical/clinical technology business-to-business marketplace.

14. A computer program product comprising a non-transitory computer readable medium having stored thereon computer executable instructions, which when executed by one or more processors, cause the one or more processors to connect agents in a medical/clinical technology business-to-business marketplace, the operations comprising:
receiving first qualifying information from a first agent in the medical/clinical technology business-to-business marketplace;
receiving second qualifying information from a second agent in the medical/clinical technology business-to-business marketplace;
determining a qualifying agent selected from one of the first agent or the second agent by filtering the first qualifying information or second qualifying information; and
connecting the qualifying agent with one of the first agent or the second agent.

15. The computer program product of claim 14, where the first qualifying information or the second qualifying information comprises a medical/clinical technology area, an employment opportunity, an employment opportunity experience level, an employment opportunity education level, an employee candidate education level, an employee candidate experience level, geographic information for the first agent or the second agent, diversity information about the first agent or the second agent, a supplier technology field, a supplier type, a supplier therapeutic relevance or a combination thereof.

16. The computer program product of claim 14, where the first agent comprises a medical/clinical technology research company or a prospective medical/clinical technology employee candidate.

17. The computer program product of claim 16, where the second agent comprises a medical/clinical technology research company, a medical/clinical technology supplier or a medical/clinical technology employee candidate.

18. The computer program product of claim 17, where the computer executable instructions to determine the qualifying agent comprise computer executable instructions to match the medical/clinical technology company with a qualifying medical/clinical technology employee candidate or to match the medical/clinical technology company with a qualifying medical/clinical technology supplier.

19. The computer program product of claim 15, where the computer executable instructions to determine the qualifying agent comprise computer executable instructions to match the qualifying candidate with the first agent or the second agent based on a highest match level of the first qualifying information and the second qualifying information.

20. The computer program product of claim 15, where the computer executable instructions to connect the qualifying agent with one of the first agent or the second agent comprise computer executable instructions to provide a query inbox for the qualifying agent and the one of the first agent or the second agent to exchange information or queries related to the medical/clinical technology business-to-business marketplace.
